(19) United States
(12) Patent Application Publication
(10) Pub. No.: US 2011/0165699 A1
(43) Pub. Date: Jul. 7, 2011

(54) IRINOTECAN IMMUNOASSAY

(76) Inventors: Salvatore J. Salamone, Stockton, NJ (US); Jodi Blake Courtney, Doylestown, PA (US); Alexander Volkov, Allentown, PA (US); Hongxia Zhang, Bethlehem, PA (US); Howard Sard, Arlington, MA (US); Vishnumurthy Hegde, Chelmsford, MA (US)

(21) Appl. No.: 13/039,868

(22) Filed: Mar. 3, 2011

Related U.S. Application Data
(63) Continuation-in-part of application No. 12/633,340, filed on Dec. 8, 2009.

(51) Int. Cl. G01N 33/98; C07K 16/44; C07D 491/22; C07K 2/00; B82Y 15/00

(52) U.S. Cl. 436/501; 530/388.9; 546/48; 530/409; 977/773; 977/902

(57) ABSTRACT

Novel conjugates of the pharmaceutically active metabolite of irinotecan and novel immunogens derived from this pharmaceutically active metabolite and monoclonal antibodies generated by these immunogens which are useful in immunoassays for the quantification and monitoring of the pharmaceutically active metabolite of irinotecan in biological fluids.

### CROSS-REFERENCE TO RELATED APPLICATIONS

This application is a Continuation-in-Part of application Ser. No. 12/633,340, filed on Dec. 8, 2009, entitled “Irinotecan Immunoassay,” which is incorporated herein by reference.

### FIELD OF THE INVENTION

This invention relates to the field of immunoassays and reagents for determining the presence and/or quantifying the amount of the pharmacologically active metabolite of irinotecan in human biological fluids.

### BACKGROUND OF THE INVENTION

Cancer is a term used to describe a group of malignancies that all share the common trait of developing when cells in a part of the body begin to grow out of control. Most cancers form as tumors, but can also manifest in the blood and circulate through other tissues where they grow. Cancer malignancies are most commonly treated with a combination of surgery, chemotherapy, and/or radiation therapy. The type of treatment used to treat a specific cancer depends upon several factors including the type of cancer malignancy and the stage during which it was diagnosed.

The chemotherapeutic agent whose common chemical name is irinotecan has the formula:

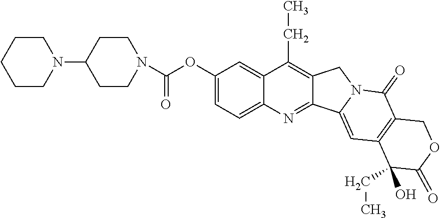

Irinotecan and its pharmaceutically acceptable salts, particularly hydrochloride is a pro-drug for administering the active SN-38 and its pharmaceutically acceptable salts. SN-38 has the formula:

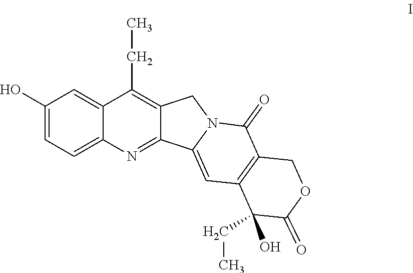

Irinotecan and its pharmaceutically acceptable salts, particularly the hydrochloride salt, is one of the more commonly used chemotherapeutic agents for treatment of first and second stage metastatic colorectal cancer in combination with 5-fluorouracil and leucovorin (Camptostar package insert, Pfizer, July 2008). Irinotecan and the active metabolite have been shown to have high inter-patient variability with CV of 25.97% and 38.91% in AUCs respectively (Sasaki, Jpn. J. Cancer, 86, 101-110, 1995) and greater than a 40 fold variation in AUCs (Canal, JCO, 14(10): 2688-2695 1996)

To monitor the pharmacokinetics of the active metabolite of irinotecan, SN-38, multiple blood samples are taken before during and after the end of infusion for which limited sampling strategies have been developed (Rouits, BJC, 99(8), 1239-45, 2008, Mathijssen, BJC, 87(2), 144-50, 2002). Dose-limiting toxicity (diarrhea and neutropenia) are a major cause of discontinuation of the drug administration. Levels of SN-38 in the plasma correlate with toxicity of neutropenia and diarrhea (Abigerges, J. Clin. Oncol, 13: 210-221, 1995; Forni, Cancer Res., 54:4347-4354, 1994). Monitoring concentrations of irinotecan's active metabolite SN 38 [the compound of formula I] in blood and adjusting to target levels would be of value in increasing efficacy and minimizing toxicity. The degree of intra- and inter-individual pharmacokinetic variability of this active metabolite, SN-38 and its pharmaceutically acceptable salts has been reported to be more than 40 fold and is impacted by many factors, including:
Organ function
Genetic regulation
Disease state
Age
Drug-drug interaction
Time of drug ingestion
Compliance

As a result of this variability, equal doses of the same drug in different individuals can result in dramatically different clinical outcomes. The effectiveness of the same dosage of irinotecan varies significantly based upon individual drug clearance and the ultimate serum drug concentration in the patient. Therapeutic drug management would provide the clinician with insight on patient variation in drug administration. With therapeutic drug management, drug dosages could be individualized to the patient, and the chances of effectively treating the cancer without the unwanted side effects would be much higher.

Routine therapeutic drug management of the active metabolite of irinotecan would require the availability of simple automated tests adaptable to general laboratory equipment. Routine therapeutic drug management of irinotecan and its active metabolite SN-38 and its pharmaceutically acceptable salts would require the availability of simple automated tests adaptable to general laboratory equipment. The use of reverse-phase high-performance liquid chromatography with fluorescence detection of irinotecan and its chemotherapeutic metabolites SN-38 in human blood and plasma has been described (Mathijssen, BJC, 87, 144-150, 2002; Sai, Biomed. Chromatogr. 16: 209-218 1995; Titier, Ther. Drug. Monit, (27)5:634-640, 2005; Sasaki Jpn. J. Cancer Res. 86: 101-110 1995). A Liquid chromatography-electrospray MS method was developed to determine irinotecan and SN-38 (Ragot, J. Chromatogr. B 736:175-184 1999) and has also been developed in human serum. These methods are labor intensive, use expensive equipment and are not amenable to routine clinical laboratory use.

Polyclonal antibodies for an ELISA measuring irinotecan and SN-38 and its pharmaceutically acceptable salts have been developed (Saita, Biol. Pharm bull. 23 (8) 911-916, 2000). The derivatives used in the haptens for antibody development did not contain whole active structure of irinotecan and SN-38. The binding partner in the assay is different from the immunogen and the structure of this binding partner has not been elucidated. Additionally this ELISA assay took overnight to generate results.

There exists no simple immunoassay for determining the presence or quantifying the amount of SN-38 in human biological fluids of patients treated with this chemotherapeutic agent.

In order to produce a simple automated test adapted to standard laboratory equipment, monoclonal antibodies specific to SN-38 and its pharmaceutically acceptable salts would be optimal. The derivatives and immunogen used in this assay must impart through these corresponding antibodies produced specific reactivity to SN-38 and without any substantial cross reactivity to therapeutically active or inactive or pharmacologically active or inactive metabolites of irinotecan. In order to be effective in monitoring drug levels, the antibodies should be specific to SN-38 (the compound of the formula I) and not cross reactive with irinotecan.

### SUMMARY OF THE INVENTION

In accordance with this invention, we have provided a reagent for an assay for the compound of Formula I or its pharmaceutically acceptable salts, which reagent has the formula.

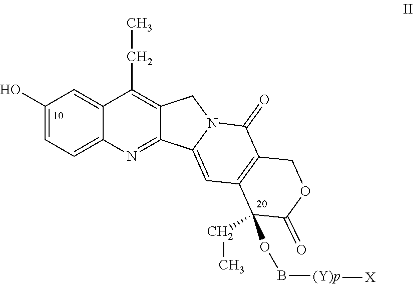

wherein B is

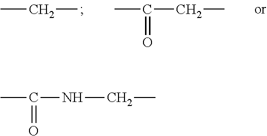

Y is an organic spacing group;
p is a an integer from 0 to 1; and
X is a terminal functional group capable of binding to a carrier,

or its pharmaceutically acceptable salts

The compound of formula II can be used both to produce an immunogen for monoclonal antibodies which can be used in an immunoassay for accurately monitoring the levels of the compound of formula I or its pharmaceutically acceptable salts. In accordance with a preferred embodiment of this invention, the compound of formula II where X is a terminal functional group capable of binding to an amino group in a polyamine containing carrier not only can be used to provide a binding partner but can also be used to provide the immunogen for an antibody for use in an immunoassay for determining the presence and/or quantifying the amount of the pharmacologically active metabolites of irinotecan(the compound of Formula I and its pharmaceutically acceptable salts. The antibody and binding partner so produced provide a simple immunoassay but can be easily carried out without extensive washing and incubating steps.

Since the immunogen for producing monoclonal antibodies, unlike the immunogen in Gaita, Biol. Pham bull. 23(8) 911-916 (2000), and the binding partner of this invention contain the whole active structure of irinotecan and the compound of formula I, this immunogen produces monoclonal antibodies specifically reactive with the compounds of formula I and its pharmaceutically acceptable salts without any cross reactivity to therapeutically or pharmacologically inactive metabolites of irinotecan and its pharmaceutically acceptable salts.

### DETAILED DESCRIPTION

In accordance with this invention, we have discovered a new reagent (i.e., the compound of formula II) which can be used to produce an immunogen and binding partner for an immunoassay to detect and quantify the amounts of the active metabolites of irinotecan, which is the compound of formula I and its pharmaceutically acceptable salts. In accordance with this invention, we have found a means for preparing both an immunogen and a binding partner to the compound of formula I without opening the functional lactone ring of the molecular structure of irinotecan and the compound of formula II. As can be seen the compound of formula II has the lactone ring structure of the compound of formula I and is produced without opening the lactone ring. In this manner a reagent is obtained for producing antibodies, particularly monoclonal antibodies and binding partners which can accurately and specifically determine the active metabolites of irinotecan via an immunoassay.

It is through the use of the compound of Formula II that the binding partner and the antibodies for an immunoassay, including reagents and kits for such immunoassay for detecting and/or quantifying the compound of formula and its pharmaceutically acceptable salts, the pharmaceutically active metabolite of irinotecan, in blood, plasma or other body fluid samples has been developed. By use of this immunoassay, the presence and amount of the compound of formula I and its pharmaceutically acceptable salts in body fluid samples, preferably a blood or plasma sample, can be detected and/or quantified. In this manner, a patient being treated with irinotecan and/or its pharmaceutically acceptable salts, can be monitored during therapy and treatment adjusted in accordance with said monitoring. By means of this invention one achieves the therapeutic drug management of irinotecan or its pharmaceutically acceptable salts in cancer patients being treated with irinotecan and/or its pharmaceutically acceptable salts as a chemotherapeutic agent.

The reagents utilized in the assay of this invention are conjugates of a carrier, preferably containing polyamine functional groups, with the compounds of formula II. These conjugates are competitive binding partners with the compound of formula I and it's pharmaceutically acceptable salts present in the sample for the binding with the antibodies of this invention. Therefore, the amount of conjugate reagent which binds to the antibody will be inversely proportional to the amount of compound of formula I and it's pharmaceutically acceptable salts in the sample. In accordance with this invention, the assay utilizes any conventional measuring means for detecting and measuring the amount of said conjugate which is bound or unbound to the antibody. Through the use of said means, the amount of the bound or unbound conjugate can be determined. Generally, the amount of compound of formula I and it's pharmaceutically acceptable salts in a sample is determined by correlating the measured amount of the bound or unbound conjugate produced by the compound of formula I and it's pharmaceutically acceptable salts in the sample with values of the bound or unbound conjugate determined from standard or calibration curve samples containing known amounts of compound of formula I, and it's pharmaceutically acceptable salts which known amounts are in the range expected for the sample to be tested. These studies for producing calibration curves are determined using the same immunoassay procedure as used for the sample.

The conjugates, as well as the immunogens, are prepared from compounds of the formula IL In the conjugates or immunogens, the carrier and the polyamine polymer are linked to ligand portions of the compounds of formula II. The ligand portions have the formula:

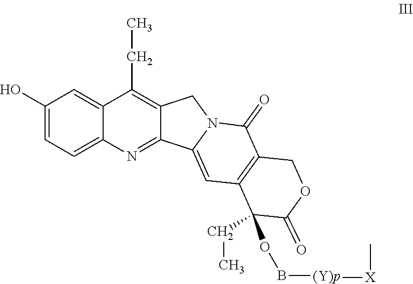

wherein X′ is —CH₂— or a functional linking group;
and Y, p and B, are as above

This ligand portion may be linked to one or more active sites on the carrier of the conjugate or the immunogen. Generally these carriers contain polymers, most preferably polyamine polymers having a reactive amino group. In forming the conjugates, X is preferably a functional group which can react with an amino group. However X can be any functional group which can react with any conventional carrier. When the compound of formula II is used to make immunogens, X in the compound of formula II is preferably any functional group capable of binding or linking to a polyamine polymer.

### Definitions

Throughout this description the following definitions are to be understood:

The term “alkylene” designates a divalent saturated straight or branch chain hydrocarbon substituent containing from one to ten carbon atoms

The terms “immunogen” and “immunogenic” refer to substances capable of eliciting, producing, or generating an immune response in an organism.

The term “conjugate” refers to any substance formed from the joining together of two parts. Representative conjugates in accordance with the present invention include those formed by the joining together of a small molecule, such as the compound of formula II and a large molecule, such as a carrier, preferably carriers which comprise a polyamine polymer, particularly a protein. In the conjugate the small molecule maybe joined or linked at one or more active sites on the large molecule. The term conjugate includes the term immunogen. In the conjugates used as reagents the carrier can be any carrier and X can be any functional group which can be linked to a carrier. In the immunogen the carrier is a polyamine polymer and X is any functional group capable of linking to a polyamine polymer.

“Haptens” are partial or incomplete antigens. They are protein-free substances, mostly low molecular weight substances, which are not capable of stimulating antibody formation, but which do react with antibodies. The latter are formed by coupling a hapten to a high molecular weight immunogenic carrier and then injecting this coupled product, i.e., immunogen, into a human or animal subject. The hapten of this invention is SN-38.

As used herein, a “spacing group” or “spacer” refers to a portion of a chemical structure which connects two or more substructures such as haptens, carriers, immunogens, labels, or tracer through a CH₂ or functional linking group. These spacer groups will be enumerated hereinafter in this application. The atoms of a spacing group and the atoms of a chain within the spacing group are themselves connected by chemical bonds. Among the preferred spacers are straight or branched, saturated or unsaturated, carbon chains. Theses carbon chains may also include one or more heteroatoms within the chain or at termini of the chains. By “heteroatoms” is meant atoms other than carbon which are chosen from the group consisting of oxygen, nitrogen and sulfur. Spacing groups may also include cyclic or aromatic groups as part of the chain or as a substitution on one of the atoms in the chain.

The number of atoms in the spacing group is determined by counting the atoms other than hydrogen. The number of atoms in a chain within a spacing group is determined by counting the number of atoms other than hydrogen along the shortest route between the substructures being connected. A functional linking group may be used to activate, e.g., provide an available functional site on, a hapten or spacing group for synthesizing a conjugate of a hapten with a label or carrier or polyamine polymer.

An “immunogenic carrier,” as the terms are used herein, is an immunogenic substance, commonly a protein, that can join with a hapten, in this case SN-38 or the SN-38 derivatives hereinbefore described, thereby enabling these hapten derivatives to induce an immune response and elicit the production of antibodies that can bind specifically with these haptens. The immunogenic carriers and the linking groups will be enumerated hereinafter in this application. Among the immunogenic carrier substances are included proteins, glycoproteins, complex polyamino-polysaccharides, particles, and nucleic acids that are recognized as foreign and thereby elicit an immunologic response from the host. The polyamino-polysaccharides may be prepared from polysaccharides using any of the conventional means known for this preparation.

Also various protein types may be employed as a poly(amino acid) immunogenic carrier. These types include albumins, serum proteins, lipoproteins, etc. Illustrative proteins include bovine serum albumin (BSA), keyhole limpet hemocyanin (KLH), egg ovalbumin, bovine thyroglobulin (BTG) etc. Alternatively, synthetic poly(amino acids) may be utilized.

Immunogenic carriers can also include poly amino-polysaccharides, which are high molecular weight polymers built up by repeated condensations of monosaccharides. Examples of polysaccharides are starches, glycogen, cellulose, carbohydrate gums such as gum arabic, agar, and so forth. The polysaccharide also contains polyamino acid residues and/or lipid residues.

The immunogenic carrier can also be a poly(nucleic acid) either alone or conjugated to one of the above mentioned poly(amino acids) or polysaccharides.

The immunogenic carrier can also include solid particles. The particles are generally at least about 0.02 microns (μm) and not more than about 100 μm, and usually about 0.05 μm to 10 μm in diameter. The particle can be organic or inorganic, swellable or non-swellable, porous or non-porous, optimally of a density approximating water, generally from about 0.7 to 1.5 g/mL, and composed of material that can be transparent, partially transparent, or opaque. The particles can be biological materials such as cells and microorganisms, including non-limiting examples such as erythrocytes, leukocytes, lymphocytes, hybridomas, Streptococcus, Staphylococcus aureus, E. coli, and viruses. The particles can also be comprised of organic and inorganic polymers, liposomes, latex, phospholipid vesicles, or lipoproteins.

“Poly(amino acid)” or “polypeptide” is a polyamide formed from amino acids. Poly(amino acids) will generally range from about 2,000 molecular weight, having no upper molecular weight limit, normally being less than 10,000,000 and usually not more than about 600,000 daltons. There will usually be different ranges, depending on whether an immunogenic carrier or an enzyme is involved.

A “peptide” is any compound formed by the linkage of two or more amino acids by amide (peptide) bonds, usually a polymer of a-amino acids in which the α-amino group of each amino acid residue (except the NH₂ terminus) is linked to the α-carboxyl group of the next residue in a linear chain. The terms peptide, polypeptide and poly(amino acid) are used synonymously herein to refer to this class of compounds without restriction as to size. The largest members of this class are referred to as proteins.

A “label,” “detector molecule,” or “tracer” is any molecule which produces, or can be induced to produce, a detectable signal. The label can be conjugated to an analyte, immunogen, antibody, or to another molecule such as a receptor or a molecule that can bind to a receptor such as a ligand, particularly a hapten. Non-limiting examples of labels include radioactive isotopes, enzymes, enzyme fragments, enzyme substrates, enzyme inhibitors, coenzymes, catalysts, fluorophores, dyes, chemiluminescers, luminescers, or sensitizers; a non-magnetic or magnetic particle, a solid support, a liposome, a ligand, or a receptor.

The term “antibody” refers to a specific protein binding partner for an antigen and is any substance, or group of substances, which has a specific binding affinity for an antigen to the exclusion of other substances. The generic term antibody subsumes polyclonal antibodies, monoclonal antibodies and antibody fragments.

The term “derivative” refers to a chemical compound or molecule made from a parent compound by one or more chemical reactions.

The term “carrier” refers to solid particles and/or polymeric polymers such as immunogenic polymers such as those mentioned above. Where the carrier is a solid particle, the solid particle may be bound, coated with or otherwise attached to the polymeric material which preferably is a polyamine polymer to provide one or more reactive sites for bonding to the terminal functional group X in the compounds of the formula II.

The term “reagent kit,” or “test kit,” refers to an assembly of materials that are used in performing an assay. The reagents can be provided in packaged combination in the same or in separate containers, depending on their cross-reactivities and stabilities, and in liquid or in lyophilized form. The amounts and proportions of reagents provided in the kit can be selected so as to provide optimum results for a particular application. A reagent kit embodying features of the present invention comprises antibodies specific for the compound of formula I. The kit may further comprise ligands of the analyte and calibration and control materials. The reagents may remain in liquid form or may be lyophilized.

The phrase “calibration and control materials” refers to any standard or reference material containing a known amount of a drug to be measured. The concentration of drug is calculated by comparing the results obtained for the unknown specimen with the results obtained for the standard. This is commonly done by constructing a calibration curve.

The term “biological sample” includes, but is not limited to, any quantity of a substance from a living thing or formerly living thing. Such living things include, but are not limited to, humans, mice, monkeys, rats, rabbits, horses, and other animals. Such substances include, but are not limited to, blood, serum, plasma, urine, cells, organs, tissues, bone, bone marrow, lymph, lymph nodes, synovial tissue, chondrocytes, synovial macrophages, endothelial cells, and skin.

### Reagents and Immunogens

In constructing an immunoassay, a conjugate of the compound of formula I is constructed to compete with the compound of formula I in the sample for binding sites on the antibodies. In the immunoassay of this invention, the reagents are conjugates of a carrier with the compound of formula II. In the compound of formula III the linker spacer constitutes the “—B—(Y)p-X′—” portion of this molecule. The linker X′ and the spacer “—B—(Y)p-”- are conventional in preparing conjugates and immunogens. Any of the conventional spacer-linking groups utilized to prepare conjugates and immunogens for immunoassays can be utilized in the compound of formula III. Such conventional linkers and spacers are disclosed in U.S. Pat. No. 5,501,987 and U.S. Pat. No. 5,101,015.

Among the preferred spacer groups are included the spacer groups hereinbefore mentioned. Particularly preferred spacing groups are groups such as alkylene containing from 1 to 10 carbon atoms,

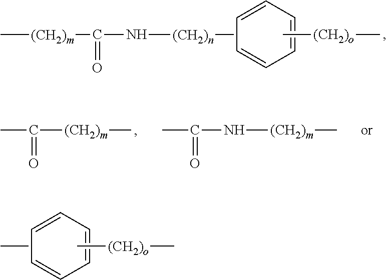

wherein m and o are integers from 0 to 6, and n is an integer from 1 to 6 with alkylene being the especially preferred spacing group In these formulae m is o, n is preferably an integer of from 1-6, most preferably 1 or 2 and o is preferably 0 or 1.

In the compound of formula III, X′ is —CH₂— or a functional group linking the spacer to the carrier, preferably to an amine group on a polymeric carrier. The group X′ is the result of the terminal functional group X in the compound of Formula II which is capable of binding to a carrier, preferably to an amino group in the polyamine polymer present in the carrier or used as the immunogen. Any terminal functional group capable of binding to a carrier, preferably capable of reacting with an amine can be utilized as the functional group X in the compound of formula II. These terminal functional groups preferably included within X are:

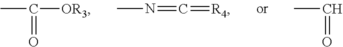

wherein R₃ is hydrogen or taken together with its attached oxygen atom forms a reactive ester and R₄ is oxygen or sulfur. The radical —N═C═R₄ can be an isocyanate or an isothiocyanate. The active esters formed by OR₃ include imidoester, such as N-hydroxysuccinamide, i-hydroxy benzotriazole and p-nitrophenyl ester. However any active ester which can react with an amine group can be used.

The carboxylic group and the active esters are coupled to the carrier or immunogenic polymer by conventional means. The amine group on the polyamine polymer, such as a protein, produces an amide group which connects the spacer to the polymer, immunogens or carrier and/or conjugates of this invention. On the other hand, carriers can be coated with a polyamine polymer to supply the amino group for linking to the ligand portion.

In the immunogens and conjugates of the present invention, the chemical bonds between the carboxyl group-containing the compound of formula I as a hapten and the amino groups on the polyamine polymer on the carrier or immunogen can be produced using a variety of methods known to one skilled in the art. It is frequently preferable to form amide bonds. Amide bonds are formed by first activating the carboxylic acid moiety of the hapten in the compounds of formula II by reacting the carboxy group with a leaving group reagent (e.g., N-hydroxysuccinimide, 1-hydroxybenzotriazole, p-nitrophenol p-nitrophenyl chloroformate o- and the like). An activating reagent such as dicyclohexylcarbodiimide, diisopropylcarbodiimide and the like can be used. The activated form of the carboxyl group in the hapten of formula II is then reacted with a buffered solution containing the protein carrier. Various methods of conjugating haptens and carriers are also disclosed in U.S. Pat. No. 3,996,344 and U.S. Pat. No. 4,016,146, which are herein incorporated by reference.

Where X is a terminal isocyanate or isothiocyanate radical in the compound of formula II, these radicals when reacted with the free amine of a polyamine polymer produce the conjugate or the immunogen of the hapten of formula III where X′ is,

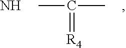

In the ligand of formula III X′ functionally connects the hapten with the amino group on the polyamine containing carrier or on the immunogenic polypeptide.

Where X, in the compounds of formula II is an aldehyde group these compounds may be connected to the amine group of the polyamine polypeptide or carrier through an amine linkage by reductive amination. Any conventional method of condensing an aldehyde with an amine such as through reductive amination can be used to form this linkage. In this case, X′ in the ligand portions of formula III.

In preparing the conjugate from the compound of formula I, the hydroxy group at the 10-position on the compound of formula I is first protected. In accordance with this invention it has been found that the best type of protection is by utilizing an ether protecting group. Since the ether protecting group can protect this hydroxy group through its conversion by various reactions to the conjugate of formula II for use as an immunogen or binding partner without destroying the lactone ring on the compound of formula I. Any conventional method of protecting a hydroxy group by etherification can be utilized to protect the hydroxy group in the compound of formula I. Any of the conventional ethers for protecting a hydroxy group can be utilized to protect the hydroxy group at the 10-position in the compound of Formula I while leaving the other hydroxy group at the 20 position in the compound of Formula I free for further reaction. Among the preferred hydroxy protecting groups are the alkyl haloalkyl ethers or the silyl ethers, alkyl ethers. Among the preferred ethers are included are methyl chloromethyl ether, methoxy methyl ether, methoxy ethyl ether and (2-(trimethylsilyl)ethoxymethyl ether. Any hydroxy protecting group such as the ester protecting groups can be utilized, which include the allyl orthoformate ester groups, can be utilized to protect the 10-hydroxy group in the compound of formula I. However these esters will not produce the beneficial results achieved by the ether protecting groups. The ether hydroxy protecting groups as well as the ester hydroxy protecting groups can be easily removed at a later stage in the process for or after forming the conjugates of formula II.

After protecting the 10-hydroxy group, the 10-protected hydroxy compound of formula I can be reacted with a leaving group which attaches to the free hydroxy group at the 20-position in the compound of formula I so as to activate this free hydroxy group for reaction with the compound of formula V-A or VI-A as described herein below. In this reaction, the free hydroxy group at the 20-position in compound of formula I with the 10-protected hydroxy group is then reacted with a leaving group to activate the free hydroxy group in the compound of formula I. The compound so activated is then reacted with a halide of the formula:

halo-CH₂—(Y)ₚ—X V-A

wherein p, Y and X are as above,

to produce derivative of the compound of formula II where B is —CH₂ with the 10-hydroxy is protected. In forming these derivatives, any conventional means of reacting an alcohol with a halide to form an ether can be utilized in condensing the compound of formula V-A with the activated hydroxy position on the compound of formula I. The use of a halide in the compound of formula V-A provides an efficient means for forming ether by condensing with the alcohol. On the other hand, where the compound of formula V-A contains functional groups, which may interfere with this reaction to form these derivatives, these functional groups can be protected by means of suitable protecting groups which can be removed after this reaction as described hereinabove.

The above derivatives of formula II where B is

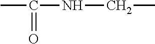

are produced by reacting the free hydroxy group on the 10-hydroxy protected compound of formula I with an amino compound of the formula:

NH₂—CH₂—(Y)p-X VI
wherein X, Y and p are as above,

Any conventional means of condensing a reactive hydroxy group with the amine group in the compound of formula VI can be used in carrying out this condensation reaction. Prior to this reaction, the reactive groups on the compound of formula VI can be protected as described hereinabove with a conventional protecting group. These protecting groups can be removed after this amine condensation by conventional means. Also the 10-protected hydroxy group can be removed after the amine condensation.

The compound of formula I when B is in

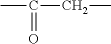

is produced by treating the free hydroxy group at the 20-position of the 10-hydroxy protected compound of formula I with a carboxylic an acid compound of the formula.

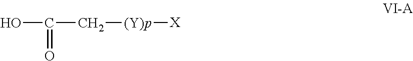

where X, Y and p are as above;

Any conventional method of esterification can be used in this reaction to form the compound of formula II where B is

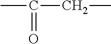

In cases where the compounds of formula V-A, VI and VI-A contain a primary or secondary amino group as well as the carboxyl group, it is necessary to use an amine or ester protecting group during the reactions to form the conjugates. Typically, the amines are protected by forming the corresponding N-trifluoroacetamide, N-tertbutyloxycarbonyl urethane (N-t-BOC urethane), N-carbobenzyloxy urethane or similar structure. Once the coupling reaction to the immunogenic polymer or carrier has been accomplished, as described above, the amine or the ester protecting group can be removed using reagents that do not otherwise alter the structure of the immunogen or conjugate. Such reagents and methods are known to one skilled in the art and include weak or strong aqueous or anhydrous acids, weak or strong aqueous or anhydrous bases, hydride-containing reagents such as sodium borohydride or sodium cyanoborohydride and catalytic hydrogenation.

The compound of formula II can be converted into the immunogens and/or the conjugate reagents of this invention by reacting these compounds with a carrier, preferably a polyamine polypeptide or a carrier coated with a polyamine polypeptide as described above. The same polypeptide can be utilized as the carrier and as the immunogenic polymer in the immunogen of this invention provided that polyamines or polypeptides are immunologically active. However, to form the conjugates used as reagents in the immunoassay, these polymers need not produce an immunological response as needed for the immunogens. In accordance with this invention, the various functional group represented by X in the compounds of formula II can be conjugated to the carrier by conventional means of attaching a functional group to a carrier. In accordance with a preferred embodiment, in the compounds of formula II, X is a carboxylic acid group or an activated carboxyl group.

### Antibodies

The present invention also relates to novel monoclonal antibodies to the compound of formula I produced by utilizing the aforementioned immunogens. In accordance with this invention it has been found that these antibodies produced in accordance with this invention are reactive with the compound of formula I and do not substantially react with irinotecan, its pharmaceutically acceptable salts or with pharmacologically inactive metabolites of irinotecan which would interfere with immunoassays for the compound of formula I.

The present invention relates to novel monoclonal antibodies to SN-38. The antisera of the invention can be conveniently produced by immunizing host animals with the immunogens of this invention. Suitable host animals include rodents, such as, for example, mice, rats, rabbits, guinea pigs and the like, or higher mammals such as goats, sheep, horses and the like. Initial doses, bleedings and booster shots can be given according to accepted protocols for eliciting immune responses in animals. Through periodic bleeding, the blood samples of the immunized mice were observed to develop an immune response against the compound of formula I utilizing conventional immunoassays. These methods provide a convenient way to screen for hosts and antibodies which are producing antisera having the desired activity. The antibodies were also screened against irinotecan and antibodies were produced which showed no substantial binding to irinotecan.

Monoclonal antibodies are produced conveniently by immunizing Balb/c mice according to the schedule followed by injecting the mice with additional immunogen i.p. or i.v. on three successive days starting three days prior to the cell fusion. Other protocols well known in the antibody art may of course be utilized as well. The complete immunization protocol detailed herein provided an optimum protocol for serum antibody response for the antibody to the compound of formula I.

B lymphocytes obtained from the spleen, peripheral blood, lymph nodes or other tissue of the host may be used as the monoclonal antibody producing cell. Most preferred are B lymphocytes obtained from the spleen. Hybridomas capable of generating the desired monoclonal antibodies of the invention are obtained by fusing such B lymphocytes with an immortal cell line, which is a cell line that which imparts long term tissue culture stability on the hybrid cell. In the preferred embodiment of the invention the immortal cell may be a lymphoblastoid cell or a plasmacytoma cell such as a myeloma cell. Murine hybridomas which produce SN-38 monoclonal antibodies are formed by the fusion of mouse myeloma cells and spleen cells from mice immunized with the aforementioned immunogenic conjugates. Chimeric and humanized monoclonal antibodies can be produced by cloning the antibody expressing genes from the hybridoma cells and employing recombinant DNA methods now well known in the art to either join the subsequence of the mouse variable region to human constant regions or to combine human framework regions with complementary determining regions (CDR's) from a donor mouse or rat immunoglobulin. An improved method for carrying out humanization of murine monoclonal antibodies which provides antibodies of enhanced affinities is set forth in International Patent Application WO 92/11018.

Polypeptide fragments comprising only a portion of the primary antibody structure may be produced, which fragments possess one or more immunoglobulin activities. These polypeptide fragments may be produced by proteolytic cleavage of intact antibodies by methods well known in the art, or by inserting stop codons at the desired locations in expression vectors containing the antibody genes using site-directed mutageneses to produce Fab fragments or (Fab′)₂ fragments. Single chain antibodies may be produced by joining VL and VH regions with a DNA linker (see Huston et al., Proc. Natl. Acad. Sci. U.S.A., 85:5879-5883 (1988) and Bird et al., Science, 242:423-426 (1988))

The antibodies of this invention are selectively reactive with the compound of Formula I and it's pharmaceutically acceptable salts and not irinotecan. In addition, the preferred antibodies do not have any substantial cross-reactivity with pharmaceutically inactive metabolites of irinotecan or its pharmaceutically acceptable salts. By substantial cross-reactivity it is meant that the antibodies of this invention have a cross reactivity with irinotecan, its pharmaceutically acceptable salts or the pharmaceutically inactive metabolites of irinotecan each of 20% or less, preferably 10% or less, said cross reactivity being based on their reactivity with the compound of Formula I and its pharmaceutically acceptable salts.

### Immunoassays

In accordance with this invention, the conjugates and the antibodies generated from the immunogens of the compound of formula II can be utilized as reagents for the determination of the compound of formula I in patient samples. This determination is performed by means of an immunoassay. Any immunoassay in which the reagent conjugates formed from the compound of formula II compete with the compound of formula I in the sample for binding sites on the antibodies generated in accordance with this invention can be utilized to determine the presence of the compound of formula I in a patient sample. The manner for conducting such an assay for the compound of formula I in a sample suspected of containing SN-38, comprises combining an (a) aqueous medium sample, (b) an antibody to the compound of formula I generated in accordance with this invention and (c) the conjugates formed from the compound of formula II. The compound of formula I in the sample can be determined by measuring the inhibition of the binding to the specific antibody of a known amount of the conjugate added to the mixture of the sample and antibody. The result of the inhibition of such binding of the known amount of conjugates by the unknown sample is compared to the results obtained in the same assay by utilizing known standard solutions of the compound of formula I. In determining the amount of the compound of formula I in an unknown sample, the sample, the conjugates formed from the compounds of formula II and the antibody may be added in any order.

Various means can be utilized to measure the amount of conjugate formed from the compound of formula II bound to the antibody. One method is where binding of the conjugates to the antibody causes a decrease in the rate of rotation of a fluorophore conjugate. The amount of decrease in the rate of rotation of a fluorophore conjugate in the liquid mixture can be detected by the fluorescent polarization technique such as disclosed in U.S. Pat. No. 4,269,511 and U.S. Pat. No. 4,420,568.

On the other hand, the antibody can be coated or absorbed on nanoparticles so that when these particles react with the compound of formula I and conjugates formed from the compounds of formula II these nanoparticles form an aggregate. However, when the antibody coated or absorbed on nanoparticles react with the SN-38 in the sample, the SN-38 from the sample bound to these nanoparticles does not cause aggregation of the antibody nanoparticles. The amount of aggregation or agglutination can be measured in the assay mixture by absorbance.

On the other hand, these assays can be carried out by having either the antibody or the compounds of formula II attached to a solid support such as a microtiter plate or any other conventional solid support including solid particles. Attaching antibodies and proteins to such solid particles is well known in the art. Any conventional method can be utilized for carrying out such attachments. In many cases, in order to aid measurement, labels may be placed upon the antibodies, conjugates or solid particles, such as radioactive labels or enzyme labels, as aids in detecting the amount of the conjugates formed from the compound of formula II which is bound or unbound with the antibody. Other suitable labels include chromophores, fluorophores, etc.

As a matter of convenience, assay components of the present invention can be provided in a kit, a packaged combination with predetermined amounts of new reagents employed in assaying for SN-38. These reagents include the antibody of this invention, as well as, the conjugate formed from the compounds of formula II. In carrying out an immunoassay in accordance with this invention the radicals p, X, Y and B in the reagent and the immunogen which forms the antibody used in a given immunoassay can be the same or be a different substituent within the groups defined for each of theses radicals. Therefore while the definitions of the radicals p, X, Y, and B are the same for the conjugate reagent and the immunogen, the particular substituent which these radicals represent for the immunogen and the conjugate reagent in a given assay may be different.

In addition to these necessary reagents, additives such as ancillary reagents may be included, for example, stabilizers, buffers and the like. The relative amounts of the various reagents may vary widely to provide for concentrations in solution of the reagents which substantially optimize the sensitivity of the assay. Reagents can be provided in solution or as a dry powder, usually lyophilized, including excipients which on dissolution will provide for a reagent solution having the appropriate concentrations for performing the assay.

### EXAMPLES

In the Examples, the following abbreviations are used for designating the following:
SN-38 7-ethyl-10-hydroxy-camptotheen
EA Ethyl alcohol
MOM-Cl Methyl chloromethyl ether
HATU O-(7-Azabenzotirazol-1-yl)-N,N,N′,N′=tetramethyluronium hexafluorophosphate
DMF Demethylformamide
DMSO Dimethylsufoxide
MeOH Methanol
EtOAc Ethyl acetate
DCM Dichloromethane
DMAP Dimethylaminopyridine
Et₃N Triethyl amine
NHS N-hydroxy-succinimide
EDC 1-(3-dimethylaminopropyl)-3-ethylcarbodiimide hydrochloride
TLC Thin Layer Chromatrography
KLH Keyhole Limpet Hemocyanin
ANS 8-Anilino-1-naphthalenesulfonic acid
i.p. Intraperitoneal
HRP Horse radish-peroxidase
TMB 3,3′,5,5′-Tetramethylbenzidine
TRIS Tris(hydroxymethyl)aminomethane hydrochloride
BSA Bovine serum albumin
BTG Bovine thyroglobulin
PBS Phosphate buffered saline
HEPES 4-(2-Hydroxyethyl)piperazine-1-ethanesulfonic acid
ANS 8-Anilino-1-naphtalenesulfonic acid
TMB 3,3′,5,5′-Tetramethylbenzidine
diH₂O deionized water

The phosphate buffer composition has an aqueous solution containing
15.4 mM Sodium phosphate dibasic (Na₂HPO₄)
4.6 mM Sodium phosphate monobasic (NaH₂PO₄)
pH=7.2±0.10

In the Examples, Scheme 1 Scheme 2, Scheme 3 and Scheme 4 below set forth the specific compounds prepared and referred to by numbers in the Examples. The schemes are as follows:

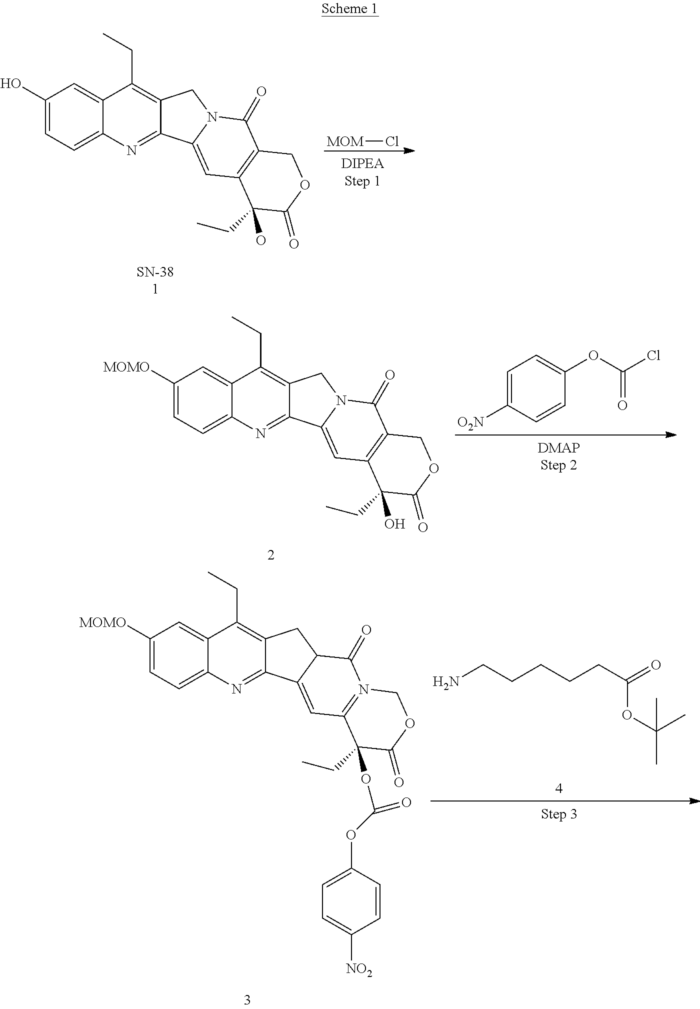

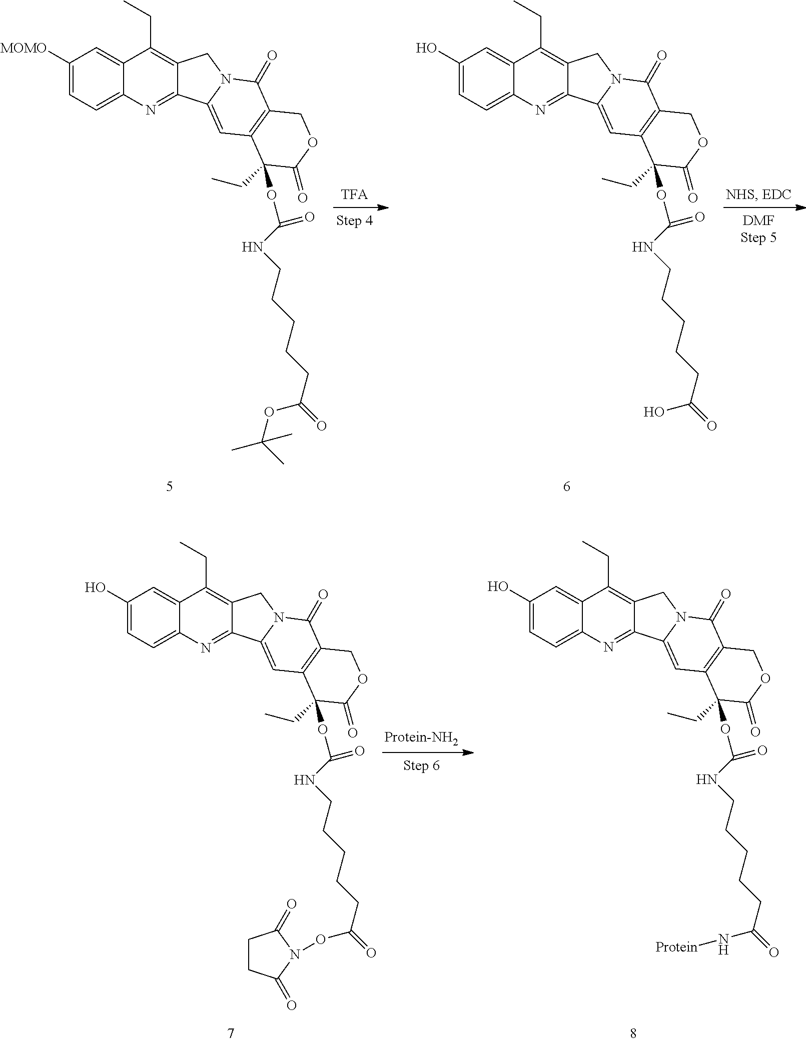

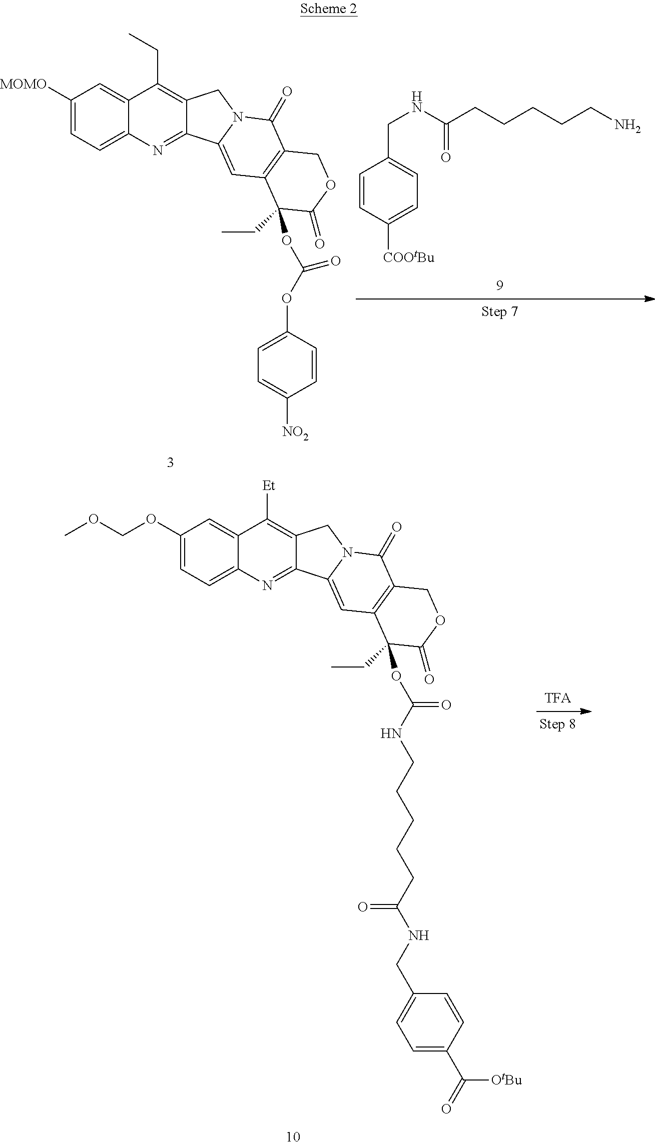

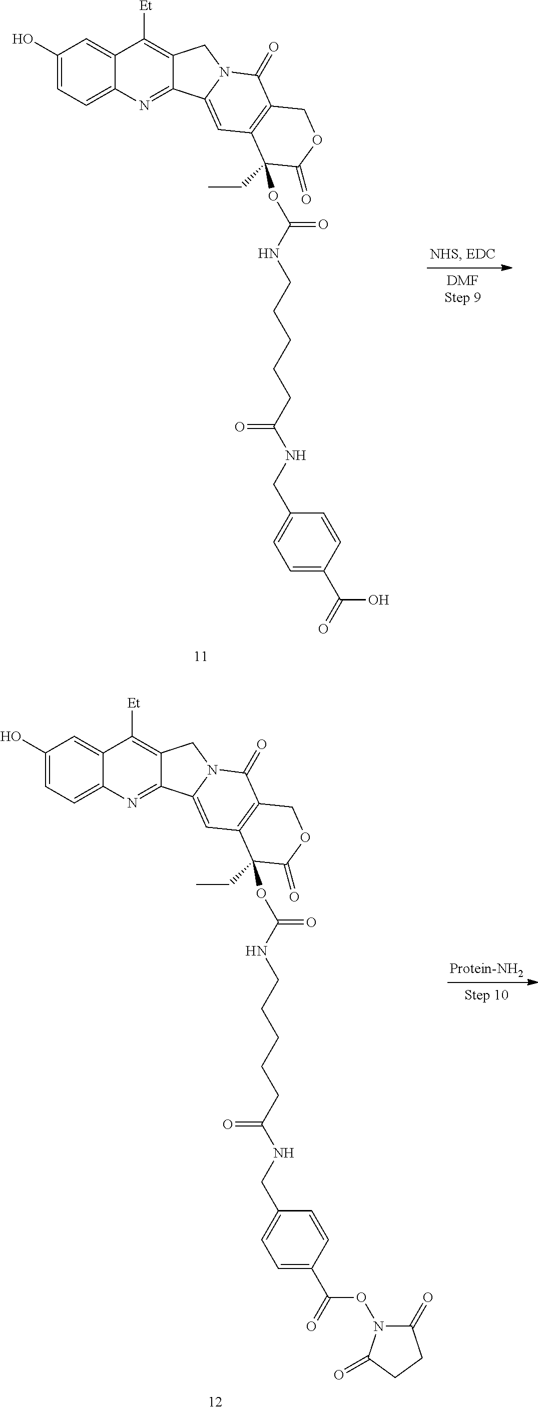

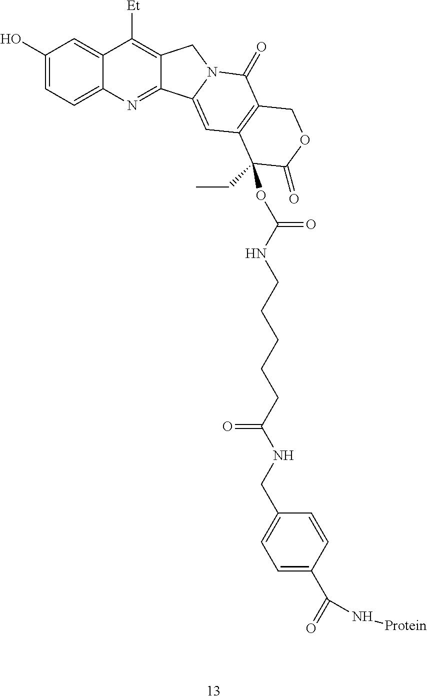

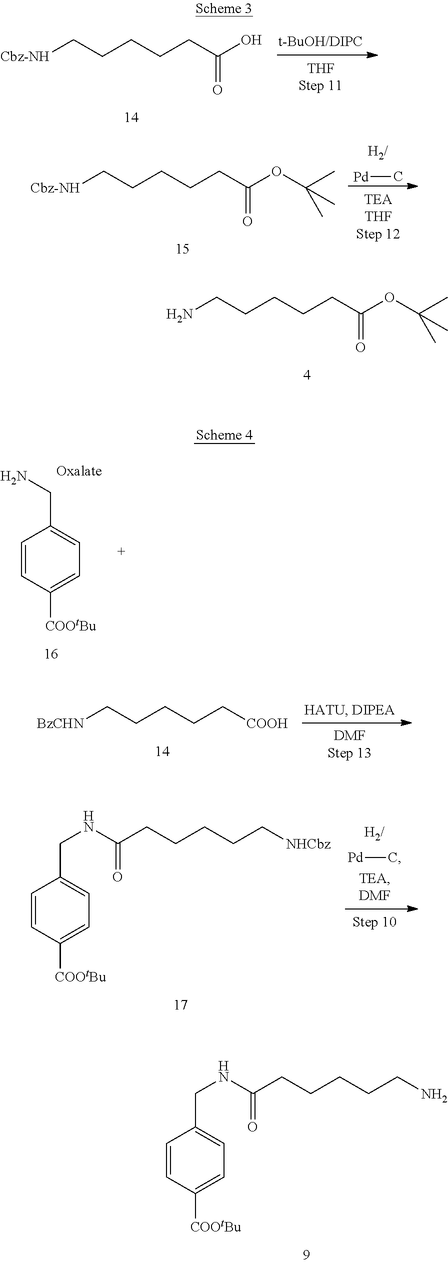

### Example 1

### Preparation of SN-38 Aminocaproic Carbamate Derivative [6] (Scheme 1)

Compound [1] (SN-38) (2.0 g, 5.10 mmol) was suspended in methylene chloride (30 mL). Diisopropylethyl amine (DIPEA) (3.30 g, 25.5 mmol) was added followed by the addition of MOM-Cl (2.05 g, 25.5 mmol). The reaction mixture was stirred at ambient temperature for 24 h resulting in a mixture containing the 10-OH methoxymethyl SN-38 derivative [2]. This mixture was diluted with methylene chloride, and washed with water, brine, dried (sodium sulfate) and filtered. The methylene chloride was removed under reduced pressure to isolate the 10-OH methoxymethyl SN-38 derivative as a yellow solid. To remove impurities this yellow solid was triturated with a 1:1 (400 mL) mixture of ether and methylene chloride to isolate pure compound [2] (2.11 g, 95%).

Compound [2] (2.20 g, 5.04 mmol) was dissolved in anhydrous methylene chloride (140 mL) and under nitrogen at 0° C. p-nitrophenyl chloroformate (3.05 g, 15.12 mmol) was added, followed by portionwise addition of 4-dimethylaminopyridine (DHAP). After stirring the mixture for 1 h at ambient temperature the reaction to produce p-Nitrophenyl-O-carbonyl-[20-(S)—O-7-ethyl-10-methoxymethyloxy camptothecin [3] was complete. The reaction mixture was diluted with methylene chloride, washed with 0.1 N HCl, water, and brine. The methylene chloride organic phase was dried (sodium sulfate) filtered and evaporated resulting in [3] as a yellow solid. To remove impurities the yellow solid was triturated with ether to obtain the desired compound [3] (2.90 g, 96%).

The amine [4] tert-Butyl 6-aminohexanoate (1.08 g, 5.78 mmol) prepared as in example 3, was added to a solution of compound [3] (2.90 g, 4.82 mmol) in anhydrous DMF (40 mL) under nitrogen. The resulting solution was stirred at ambient temperature for 3 h to produce the tert-Butyl N-carbonyl-[20-(S)—O-7-ethyl-10-methoxymethyloxycamptothecin]-6-aminocaproate [5]. The reaction mixture was poured onto water and extracted with ethyl acetate. The ethyl acetate organic phase was washed with water, brine, dried (sodium sulfate), filtered and evaporated to obtain the product [5] as a yellow solid. To remove impurities the yellow solid was triturated with 20% ether/hexanes to isolate the pure material [5] (2.51 g, 80%).

To a solution of compound [5] (2.5 g, 3.84 mmol) in anhydrous methylene chloride (10 mL) at 0° C. under nitrogen trifluoroacetic acid (TFA) (10 mL) was added dropwise, stirred at 0° C. for 10 min, then at ambient temperature overnight to remove the t-butyl and the methoxymethyloxy groups from [5] to produce compound [6]. The methylene chloride was removed under reduced pressure resulting in [6] as a yellow solid which was purified by flash chromatography using 2-8% methanol/chloroform/0.1% acetic acid as eluent. The fractions containing pure product were combined and the solvents were removed to isolate a yellow solid which was triturated with ethyl acetate to obtain the pure compound [6] (1.48 g, 70%).

### Example 2

### Preparation of SN-38 Benzylcarbamoyl Derivative [11] (Scheme 2)

A solution of amine [9] produced as in example 4 (578 mg, 1.80 mmol) in dry DMF (6 mL) was added dropwise to a solution of [3] (846 mg, 1.39 mmol) in DMF (8 mL). The reaction mixture was stirred at ambient temperature for 3 h producing the protected benzylcarbamoyl derivative [10]. The reaction was quenched by the addition of water and extracted with ethyl acetate. The ethyl acetate organic layer was washed with brine, dried (sodium sulfate), filtered and evaporated. The crude product was purified by flash chromatography using ethyl acetate/methanol 99:1 to 98:2 to afford compound [10] (832 mg, 77%).

Trifluoroacetic acid (TFA) (2 mL) was added to a solution of [10] (664 mg, 0.84 mmol) at 0° C. The reaction mixture was allowed to warm to ambient temperature and was stirred overnight to hydrolize the protecting groups from [10] to produce compound [11]. The solvent methylene chloride was evaporated and co-evaporated with toluene. The residue was purified by flash chromatography using methylene chloride/methanol 95:5 to 92:8. Evaporation of the solvents followed by trituration with ether afforded compound [11].

### Example 3

### Preparation of tert-Butyl 6-aminohexanoate [4] (Scheme 3)

Tert-Butyl 6-aminohexanoate was prepared for the synthesis of tert-Butyl N-carbonyl-[20-(S)—O-7-ethyl-10-methoxymethyloxycamptothecin]-6-aminocaproate, scheme 1, compound [5], example 1. A solution of diisopropylcarbodiimide (DIPC) (5.24 g, 41.52 mmol) in anhydrous methylene chloride (16 mL) was added to a mixture of the acid [14] (10.0 g, 37.70 mmol), 4-(Dimethylamino)pyridine (DMAP) (90.94 g, 7.70 mmol), and anhydrous t-butylalcohol (tBuOH) (5.60 g, 75.40 mmol) in anhydrous methylene chloride (50 mL) at 0° C. under nitrogen. The reaction mixture was warmed to room temperature and stirred overnight to produce tert-Butyl 6-(benzyloxycarbonylamino)hexanoate [15]. The solid precipitated from the reaction mixture was removed by filtration from the solution containing the product, and washed with methylene chloride. The filtrate was evaporated to obtain the crude product [15]. The crude product was purified by flash chromatography using 10-30% ethyl acetate/hexanes as eluent to isolate the compound [15] as a colorless oil (11.4 g, 97%).

¹H NMR (CDCl₃): δ ppm 7.35 (m, 5H), 5.08 (s, 2H), 4.75 (bt, 1H), 3.17 (q, J=6.90 Hz, 2H), 2.17 (t, J=7.40 Hz, 2H), 1.20-1.60 (m, 15H).

Compound [15] (11.4 g, 36.50 mmol) was dissolved in THF (240 mL) to which was added triethylamine (TEA) (1 mL) followed by palladium carbon catalyst (Pd—C) (10 wt %, 0.7 g). This mixture was hydrogenated at normal pressure (balloon) for 20 h to remove the carbobenzyloxy (Cbz) amine protecting group. The reaction mixture was filtered through a pad of Celite, washed with THF and the filtrate was evaporated to obtain the desired amine [4] (5.90 g, 87%) as a pale yellow oil.

¹H NMR (CDCl₃): δ ppm 2.68 (t, J=6.90 Hz, 2H), 2.17 (t, J=7.40 Hz, 2H), 1.20-1.60 (m, 15H).

### Example 4

### Preparation of 4-[(6-Amino-hexanoylamino)-methyl]-benzoic acid tert-butyl ester [9] (Scheme 4)

The 4-[(6-Amino-hexanoylamino)-methyl]-benzoic acid dertivative was prepared for the synthesis of 7-Ethyl-10-hydroxy-20-(5-[4-carboxy-benzylcarbamoyl]-pentylcarbamoyl)camptothecin, compound [11], scheme 2. The coupling reagent HATU (1.72 g, 4.77 mmol) was added to a mixture of [14] (1.0 g, 3.77 mmol), [16] (1.18 g, 3.98 mmol) and N,N-Diisopropylethylamine (DIPEA) (3.2 mL, 18.92 mmol) in dry DMF (20 mL) at 0° C. The reaction mixture was stirred at ambient temperature overnight. An additional amount of HATU (1.72 g, 4.77 mmol) was added and the reaction mixture was stirred at ambient temperature for 24 h to produce [17]. The reaction mixture was partitioned between water and ethyl acetate. The ethyl acetate organic layer was successively washed with 1N HCl, saturated sodium bicarbonate, water, and saturated brine, and dried (sodium sulfate). After filtering the organic solvent was evaporated to give 1.98 g of [17] which was used as such for the next step.

¹H NMR (300 MHz, CDCl₃): δ 7.93 (d, J=8.1 Hz, 2H), 7.26-7.37 (m, 7H), 5.85 (bs, 1H), 5.06 (s, 2H), 4.81 (bs, 1H), 4.46 (d, J=5.8 Hz, 2H), 3.14-3.21 (m, 2H), 2.22 (t, J=7.1 Hz, 2H), 1.29-1.81 (m, 15 H). APCI+=455.

A mixture of [17] (1.98 g) in anhydrous THF (36 mL) containing triethylamine (0.15 mL) and 10% Pd—C (100 mg) was reacted under a hydrogen atmosphere (1 atmosphere). After 1 day, 10% Pd—C (100 mg) and 5 drops of water were added and hydrogenation continued for 1 day. The contents of the flask were filtered through a pad of Celite and evaporation of the filtrate afforded [9] (1.5 g, quantitative).

¹H NMR (300 MHz, CDCl₃): δ 7.91 (d, J=8.2 Hz, 2H), 7.29 (d, J=8.2 Hz, 2H), 6.16 (t, J=5.8 Hz, 1H), 4.44 (d, J=5.8 Hz, 2H), 2.79 (s, 2H), 2.70-2.75 (m, 2H), 2.20-2.26 (m, 2H), 1.29-1.69 (m, 15H). APCI+=321.

### Example 5

### General Method for Preparing NHS Activated Drug Derivatives from the Corresponding Acids [6] & [11]

SN-38 acid derivatives were activated with EDC and NHS to produce the NHS activated ester of SN-38 for eventual conjugation to proteins (examples 6, 7a and 7b).

### Example 5a

### Preparation of NHS Activated Ester SN-38 Aminocaproic Carbamate Derivative [7]

SN-38 derivative [6], example 1, scheme 1, (91.8 mg) was dissolved in 10 mL of DMF to which was added NHS (56.1 mg) and EDC (92.8 mg). The reaction mixture was stirred for 20 hours at room temperature under a nitrogen atmosphere to produce the NHS activated ester of SN-38 [7]. The reaction mixture was used directly in examples 6 and 7a.

### Example 5b

### Preparation of NHS Activated Ester SN-38 Benzylcarbamoyl Derivative [12]

SN-38 derivative [11], example 2, scheme 2 (60.5 mg) was dissolved in 6 mL of DMF to which was added NHS (25.7 mg) and EDC (43.5 mg). The reaction mixture was stirred for 20 hours at room temperature under a nitrogen atmosphere to produce the NHS activated ester of SN-38 [12]. The reaction mixture was used directly in example 7b.

### Example 6

### Preparation of KLH Immunogen with Activated Hapten [7]

A protein solution of KLH was prepared by dissolving 300 mg of KLH in 19.6 mL of phosphate buffer (50 mM, pH 7.5) and then slowly adding 39.2 mL of DMSO while stirring the protein solution of KLH on ice, followed by addition of 5.1 mL of NHS activated SN-38 derivative [7] prepared in Example 5a. The reaction mixture of KLH and activated SN-38 derivative [7] was allowed to stir for 20 hours at room temperature to produce an SN-38-KLH conjugate [8]. The SN-38-KLH conjugate was then purified by dialysis against 50% DMSO in phosphate buffer (50 mM, pH 7.5) at room temperature. Thereafter the DMSO proportion was reduced stepwise: 40%, 30%, 20%, 10% and 0%. The last dialysis was performed against phosphate buffer at 4° C. The SN-38-KLH conjugate [8] was characterized by ultraviolet-visible spectroscopy (UV/VIS). The conjugate was diluted to a final concentration of 2 mg/mL in phosphate buffer (50 mM, pH 7.5).

### Example 7a

### Preparation of BSA Conjugate with Activated Hapten [7]

A protein solution of BSA was prepared by dissolving 990 mg BSA in phosphate buffer (50 mM, pH 7.5) for a final concentration of 50 mg/mL. 19.8 mL of DMSO was slowly added to the protein solution of BSA while stirring on ice, followed by addition of 0.9 mL of NHS activated SN-38 derivative [7] prepared in Example 5a. The amount of NHS activated SN-38 derivative [7] added to the protein solution of BSA was calculated for a 1:1 molar ratio between the derivative of SN-38 [7] and BSA. The mixture of BSA and activated SN-38 derivative [7] was allowed to stir for 18 hours at room temperature to produce the conjugate of the activated SN-38 ester [7] and BSA. This conjugate was then purified by dialysis against 50 % DMSO in phosphate buffer (50 mM, pH 7.5) at room temperature. Thereafter the DMSO proportion was reduced stepwise: 40 %, 30%, 20%, 10% and 0%. The last dialysis was performed against phosphate buffer at 4° C. The purified SN-38-BSA conjugate was characterized by UV/VIS spectroscopy.

### Example 7b

### Preparation of BSA Conjugate with Activated Hapten [12]

A protein solution of BSA was prepared by dissolving 990 mg BSA in phosphate buffer (50 mM, pH 7.5) for a final concentration of 50 mg/mL. 19.8 mL of DMSO was slowly added to the protein solution of BSA while stirring on ice, followed by addition of 1 mL of NHS activated SN-38 derivative [12] prepared in Example 5b. The amount of NHS activated SN-38 derivative [12] added to the protein solution of BSA was calculated for a 1:1 molar ratio between the derivative of SN-38 [12] and BSA. The mixture of BSA and activated SN-38 derivative [12] was allowed to stir for 18 hours at room temperature to produce the conjugate of the activated SN-38 ester [12] and BSA. This conjugate was then purified by dialysis against 50% DMSO in phosphate buffer (50 mM, pH 7.5) at room temperature. Thereafter the DMSO proportion was reduced stepwise: 40%, 30%, 20%, 10% and 0%. The last dialysis was performed against phosphate buffer at 4° C. The purified SN-38-BSA conjugate [13] was characterized by UV/VIS spectroscopy.

### Example 8a

### Preparation of Polyclonal Antibodies to SN-38 [6]

Ten female BALB/c mice were immunized i.p. with 100 μg/mouse of SN-38-KLH immunogen [8], as prepared in Example 6, emulsified in Complete Freund's adjuvant. The mice were boosted once four weeks after the initial injection with 100 μg/mouse of the same immunogen emulsified in Incomplete Freund's Adjuvant. Twenty days after the boost test bleeds containing polyclonal antibodies from each mouse were obtained by orbital bleed. The anti-serum from these test bleeds containing SN-38 antibodies were evaluated in Examples 10a and 11.

### Example 8b

### Preparation of Monoclonal Antibodies to SN-38 [6]

Mice from Example 8a that were immunized with SN-38-KLH conjugate [8] prepared in Example 6 were used to produce monoclonal antibodies. For monoclonal antibodies starting three days before the fusion, the mice were injected i.p. with 400 μg (3 days before fusion), 200 μg (2 days before fusion), and 200 μg (1 day before fusion) of SN-38-KLH conjugate [8] in PBS prepared in Example 6. Spleen cells were isolated from the selected mice and fused with 2×10⁷ SP2/0 cells with 50% polyethylene glycol 1500 according to the method of Coligan, J. E. et al., eds., Current Protocols in Immunology, 2.5.1-2.5.8, (1992), Wiley & Sons, NY. The fused cells were plated on ten 96-well plates in DMEM/F12 supplemented with 20% FetalClone I, 2% L-glutamine (100 mM) and 2% 50× HAT. Two to three weeks later, the hybridoma supernatant was assayed for the presence of anti-SN-38 antibodies by ELISA (as in example 10b). Cells from the wells that gave positive ELISA results were expanded to 24 well plates. Clones positive by ELISA were subcloned once by limiting dilution according to the method disclosed in Coligan, J. E. et al., eds., Current Protocols in Immunology, 2.5.8-2.5.17, (1992), Wiley & Sons, NY. Hybridoma culture supernatants containing monoclonal antibody from selected subclones were confirmed for SN-38 binding by a competitive ELISA (Example 11). These monoclonal antibodies were tested for SN-38 binding and cross-reactivity to irinotecan, the SN-38 prodrug, by indirect competitive microtiter plate assay as described in example

### Example 9a

### Microtiter Plate Sensitization Procedure with SN-38 [6]-BSA Conjugate

The ELISA method for measuring SN-38 concentrations was performed in polystyrene microtiter plates (Nunc MaxiSorp F8 Immunomodules) optimized for protein binding and containing 96 wells per plate. Each well was coated with SN-38 [6]-BSA conjugate (prepared as in Example 7a) by adding 300 μL of SN-38 [6]-BSA conjugate at 10 μg/mL in 0.05M sodium carbonate, pH 9.6, and incubating for three hours at room temperature. The wells were washed with 0.05M sodium carbonate, pH 9.6 and then were blocked with 375 μL of 5% sucrose, 0.2% sodium caseinate solution for 30 minutes at room temperature. After removal of the post-coat solution the plates were dried at 37° C. overnight.

### Example 9b

### Microtiter Plate Sensitization Procedure with SN-38 [11]-BSA Conjugate

The ELISA method for measuring SN-38 concentrations was performed in polystyrene microtiter plates (Nunc MaxiSorp F8 Immunomodules) optimized for protein binding and containing 96 wells per plate. Each well was coated with SN-38 [11]-BSA conjugate (prepared as in Example 7b) by adding 300 μL of SN-38 [11]-BSA conjugate at 10 μg/mL in 0.05M sodium carbonate, pH 9.6, and incubating for three hours at room temperature. The wells were washed with 0.05M sodium carbonate, pH 9.6 and then were blocked with 375 μL of 5% sucrose, 0.2% sodium caseinate solution for 30 minutes at room temperature. After removal of the post-coat solution the plates were dried at 37° C. overnight.

### Example 10a

### Antibody Screening Procedure—Titer

This procedure is to find the dilution of antibody to be tested for displacement as in Example 11. The ELISA method for screening SN-38 antibodies (produced in Examples 8a and 8b) was performed with the microtiter plates that were sensitized with SN-38-BSA conjugate prepared in Examples 9a and 9b. The antibody screening assay was performed by diluting the murine serum from test bleeds (as in Example 8a) containing polyclonal SN-38 antibodies to 1:2,000, 1:8,000,1:18,000 and 1:50,000 (volume/volume) in phosphate buffered saline containing 0.1% BSA and 0.01% thimerosal. For evaluation of monoclonal antibodies, hybridoma supernatants of Example 8b, which were found to be positive for the presence of antibody by the procedure of Example 10b were diluted 1:10, 1:100, 1:1000, etc. (volume/volume) in phosphate buffered saline containing 0.1% BSA and 0.01% thimerosal. To each well of SN-38-BSA sensitized wells (prepared in Examples 9a and 9b) 50 μL phosphate buffered saline containing 0.1% BSA and 0.01% thimerosal and 50 μL of diluted antibody were added and incubated for 10 minutes at room temperature with shaking. During this incubation antibody binds to the SN-38-BSA conjugate passively absorbed in the wells (Examples 9a and 9b). The wells of the plates were washed three times with 0.02 M TRIS, 0.9% NaCl, 0.5% Tween-80 and 0.001% thimerosal, pH 7.8 to remove any unbound antibody. To detect the amount of SN-38 antibody bound to the SN-38-BSA conjugate in the wells, 100 μL of a goat anti-mouse antibody—HRP enzyme conjugate (Jackson Immunoresearch) diluted to a specific activity (approximately 1/3000) in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody—HRP enzyme conjugate binds to SN-38 antibodies in the wells, the plates were again washed three times to remove unbound goat anti-mouse antibody—HRP enzyme conjugate. To develop a measurable color in the wells washing was followed by the addition of 100 μL of TMB (TMB Substrate, BioFx), the substrate for HRP, to develop color during a 10 minute incubation with shaking at room temperature. Following the incubation for color development, 50 μL of stop solution (1.5% sodium fluoride in diH2O) was added to each well to stop the color development and after 20 seconds of shaking the absorbance was determined at 650 nm (Molecular Devices Plate Reader). The amount of antibody in a well was proportional to the absorbance measured and was expressed as the dilution (titer) resulting in an absorbance of 1.5. Titers were determined by graphing antibody dilution of the antibody measured (x-axis) vs. absorbance 650 nm (y-axis) and interpolating the titer at an absorbance of 1.5. The titer which produced absorbance of 1.5 determined the concentration (dilution) of antibody used in the indirect competitive microtiter plate assay described in Example 11.

### Example 10b

### Antibody Screening Procedure—Monoclonal Screening

The ELISA method for screening SN-38 monoclonal antibodies (produced in example 8b) was performed with the microtiter plates that were sensitized with SN-38-BSA conjugate as described in examples 9a and 9b. To each well of SN-38-BSA sensitized wells (prepared in examples 9a and 9b) 50 μL phosphate buffered saline containing 0.1% BSA and 0.01% thimerosal and then 50 μL of monoclonal culture supernatant were added and incubated for 10 minutes at room temperature with shaking. During this incubation antibody binds to the SN-38-conjugate in the well. The wells of the plates were washed three times with 0.02 M TRIS, 0.9% NaCl, 0.5% Tween-80 and 0.001% thimerosal, pH 7.8 to remove any unbound antibody. To detect the amount of SN-38 antibody bound to the SN-38-BSA conjugate in the wells, 100 μL of a goat anti-mouse antibody—HRP enzyme conjugate (Jackson Immunoresearch) diluted 1/3000 in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody—HRP enzyme conjugate binds to SN-38 antibodies in the wells, the plates were again washed three times to remove unbound goat anti-mouse antibody—HRP enzyme conjugate. To develop a measurable color in the wells washing was followed by the addition of 100 μL of TMB (TMB Substrate, BioFx), the substrate for HRP, to develop color during a 10 minute incubation with shaking at room temperature. Following the incubation for color development, 50 μL of stop solution (1.5% sodium fluoride in diH₂O) was added to each well to stop the color development and after 10 seconds of shaking the absorbance was determined at 650 nm (Molecular Devices Plate Reader). The amount of antibody in a well was proportional to the absorbance measured. Samples with an absorbance of greater than three or more times background were designated as positive. Fifty samples with highest absorbance were expanded to 24 well plates, as described in Example 8b.

### Example 11

### Indirect Competitive Microtiter Plate Immunoassay Procedure Determining IC₅₀ and Cross-Reactivity for Antibodies to SN-38

The ELISA method for determining IC₅₀ values and cross-reactivity was performed with the microtiter plates that were sensitized with SN-38-BSA conjugates as described in Examples 9a and 9b. The analytes—SN-38 and irinotecan were diluted in diH₂O—SN-38 over a concentration range of 1 to 10,000 ng/mL for SN-38 [6]-BSA microtiter plate and 0.1 to 1,000 ng/mL for SN-38 [11]-BSA microtiter plate. Irinotecan was diluted in diH₂O over a concentration range of 2000 to 10,000 ng/mL for SN-38 [6]-BSA microtiter plates and a concentration range of 1 to 100,000 ng/mL for SN-38 [11]-BSA microtiter plates. Each of the assays were performed by incubating 50 μL of the analyte solution with 50 μL of one of the antibodies selected from the polyclonal antibodies produced in Example 8a with the immunogen of Example 6 and the monoclonal antibody produced in Example 8b. The assays were all performed by diluting the concentration of the antibodies in each of the wells to the titer determined in Example 10a. During the 10 minute incubation (at room temperature with shaking) there is a competition of antibody binding for the SN-38-BSA conjugate in the well (produced in Examples 9a and 9b) and the analyte in solution. Following this incubation the wells of the plate were washed three times with 0.02 M TRIS, 0.9% NaCl, 0.5% Tween-80 and 0.001% thimerosal, pH 7.8 to remove any material that was not bound. To detect the amount of SN-38 antibody bound to the SN-38-BSA conjugate in the wells (produced in Examples 9a and 9b), 100 μL of a goat anti-mouse antibody—HRP enzyme conjugate (Jackson Immunoresearch) diluted to a predetermined specific activity (approximately 1/3000) in PBS with 0.1% BSA, 0.05% ANS, 0.01% thimerosal, capable of binding specifically with murine immunoglobulins and producing a colored product when incubated with a substrate, in this example TMB, were added to each well. After an incubation of 10 minutes at room temperature with shaking, during which the goat anti-mouse antibody—HRP enzyme conjugate binds to SN-38 antibodies in the wells, the plates were again washed three times to remove unbound secondary conjugate. To develop a measurable color in the wells washing was followed by the addition of 100 μL of TMB (TMB Substrate, BioFx), the substrate for HRP, to develop color in a 10 minute incubation with shaking at room temperature. Following the incubation for color development, 50 μL of stop solution (1.5% sodium fluoride in diH₂O) was added to each well to stop the color development and after 20 seconds of shaking the absorbance was determined at 650 nm (Molecular Devices Plate Reader). The amount of antibody in a well was proportional to the absorbance measured and inversely proportional to the amount of SN-38 in the sample. The IC₅₀'s of SN-38 and irinotecan were determined by constructing dose-response curves with the absorbance in the wells plotted versus analyte concentration in the wells. The absorbance of the color in the wells containing analyte was compared to that with no analyte and a standard curve was generated. The IC₅₀ value for a given analyte was defined as the concentration of analyte that was required to have 50 % of the absorbance of the wells containing no analyte. The cross-reactivity was calculated as the ratio of the IC₅₀ for SN-38 to the IC₅₀ for irinotecan and expressed as a percent. When measured with this pool of antibodies, the percent cross-reactivities relative to SN-38 for irinotecan were less than or equal to 0.12%. Results for polyclonal antibodies to SN-38 are in table I below. When measured with selected monoclonal antibodies the percent cross-reactivities relative to SN-38 for irinotecan were less than 0.13%. Results for monoclonal antibodies to SN-38 are in table II.

TABLE I
TABLE I Cross-reactivity of competitive immunoassay
using polyclonal antibodies to SN-38 (Example 8a).

| | |
|---|---|
| Plates coated with SN- | Plates coated with SN- |
| 38[6]-BSA conjugate | 38[11]-BSA conjugate |
| (Example 9a) | (Example 9b) |

| Bleed # | SN-38 | Irinotecan | SN-38 | Irinotecan |
|---|---|---|---|---|
| 1 | 100% | <0.07% | 100% | 0.03% |
| 2 | 100% | <0.06% | 100% | 0.03% |
| 3 | 100% | <0.11% | 100% | 0.03% |
| 4 | 100% | <0.09% | 100% | 0.03% |
| 5 | 100% | <0.12% | 100% | 0.05% |
| 6 | 100% | <0.10% | 100% | 0.03% |
| 7 | 100% | <0.07% | 100% | 0.04% |
| 8 | 100% | <0.07% | 100% | 0.04% |
| 9 | 100% | <0.03% | 100% | 0.04% |
| 10 | 100% | <0.06% | 100% | 0.03% |
| 11 | | | 100% | <0.05% |
| 12 | | | 100% | 0.08% |
| 13 | | | 100% | <0.10% |
| 14 | | | 100% | <0.05% |
| 15 | | | 100% | <0.05% |
| 16 | | | 100% | <0.07% |
| 17 | | | 100% | <0.06% |
| 18 | | | 100% | <0.05% |
| 19 | | | 100% | <0.09% |
| 20 | | | 100% | <0.08% |

TABLE II
Cross-reactivity of competitive immunoassay using monoclonal
antibodies to SN-38 (Example 8b) assayed using microtiter
plates coated with SN-38[11]-BSA conjugate (Example 9b).

| | |
|---|---|
| Monoclonal | |
| antibody | Analyte |

| number | SN-38 | Irinotecan |
|---|---|---|
| 6D6 | 100% | 0.01% |
| 6D7 | 100% | 0.01% |
| 6D8 | 100% | 0.01% |
| 6E7 | 100% | 0.01% |
| 6E8 | 100% | 0.01% |
| 6F7 | 100% | 0.01% |
| 7H9 | 100% | 0.13% |
| 9E7 | 100% | 0.03% |
| 9E8 | 100% | 0.01% |
| 9F5 | 100% | 0.03% |
| 9H4 | 100% | 0.04% |
| 10G8 | 100% | 0.01% |

As seen from these tables, the antibodies of this invention are substantially selectively reactive with the active form of SN-38 and are not substantially cross-reactive with the inactive prodrug irinotecan.

What is claimed:

1. An immunoassay for detecting the presence of the pharmaceutically active metabolite of irinotecan having the formula 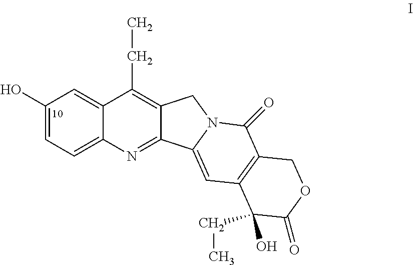
and its pharmaceutically acceptable salts, in a sample comprising providing a mixture containing a sample, an antibody selectively reactive with said metabolite and its pharmaceutically acceptable salts and a conjugate of a carrier with a ligand selected from a compound of the formula: 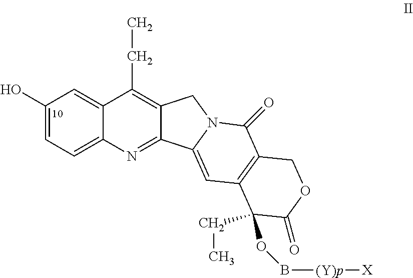
wherein B is 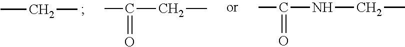
Y is an organic spacing group;
X is a functional group capable of binding to a carrier; and
p is an integer from 0 to 1;
causing the active metabolite in the sample and said conjugate to bind with said antibody and thereafter measuring the amount of said conjugate in said mixture which is bound or unbound to said antibody whereby the presence of said active metabolite and its pharmaceutically acceptable salts in the sample can be determined.

2. The process of claim 1, wherein the sample is a human sample.

3. The immunoassay of claim 1, wherein said antibody is generated from an immunogen comprising an immunogenic carrier containing a polyamine linked to a ligand selected from the group consisting of a compound of the formula: 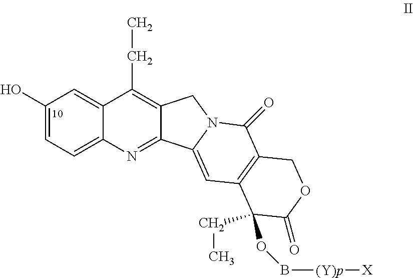
wherein p, Y and B are as above, and
X is a functional group capable of binding to an amino group of said polyamine containing immunogenic carrier.

4. The immunoassay of claim 1, wherein the antibody is attached to a solid support

5. The immunoassay of claim 4, wherein the solid support is microtiter plates

6. The immunoassay of claim 4, wherein the solid support is nanoparticles

7. The immunoassay of claim 3, wherein said antibody is a monoclonal antibody

8. The immunoassay of claim 7, wherein said antibody is derived from mice, sheep, rabbits, or rats.

9. A monoclonal antibody which reacts with the active irinotecan metabolite 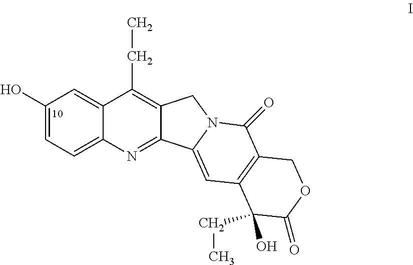
of formula I and its pharmaceutically acceptable salts and does not substantially react with irinotecan and its pharmaceutically acceptable salts.

10. The monoclonal antibody of claim 9, wherein said antibody is generated from an immunogen comprising an immunogenic polyamine containing carrier linked to a ligand selected from the group consisting of a compound of the formula: 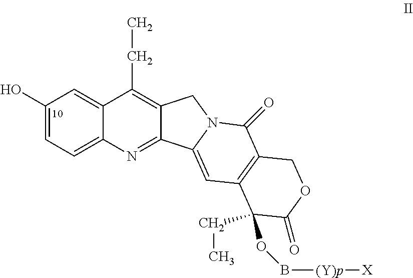
wherein B is 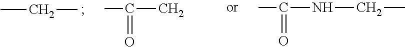
Y is an organic spacing group;
X is a functional group capable of binding to an amino group contained in said carrier; and p is an integer from 0 to 1.

11. The monoclonal antibody of claim 10 wherein said monoclonal antibody is derived from mice, sheep, rabbits, or rats.

12. A compound of the formula: 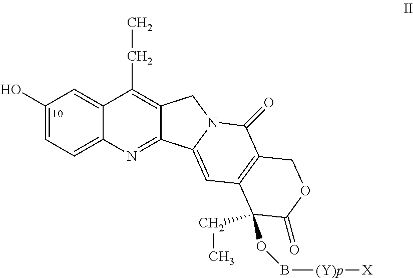
wherein B is 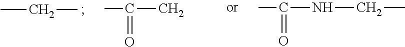
Y is an organic spacing group;
X is a functional group capable of binding to a carrier; and
p is an integer from 0 to 1 and R₁₀ is a hydrogen or a hydrolyzable hydroxy protecting group.

13. The compound of claim 12, wherein said the functional group X contains a functional group capable of binding to an amino group of a carrier containing a polyamine polymer.

14. The compound of claim 13, wherein p is 0.

15. The compound of claim 14, wherein X is 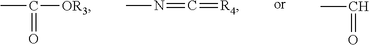
wherein R₃ is hydrogen or taken together with its attached oxygen atom forms a reactive ester and R₄ is oxygen or sulfur.

16. The compound of claim 15, wherein X is 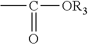
and R₃ is hydrogen.

17. The compound of claim 15, wherein X is 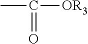
and R₃ forms a reactive ester.

18. The compound of claim 17, wherein the ester formed is a lower alkyl ester, imidoester or amidoester.

19. The compound of claim 13, wherein p is 1.

20. The compound of claim 19, wherein X is 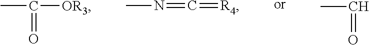
wherein R₃ is hydrogen or taken together with its attached oxygen atom forms a reactive ester and R₄ is oxygen or sulfur.

21. The compound of claim 20, wherein Y is alkylene containing from 1 to 10 carbon atoms, 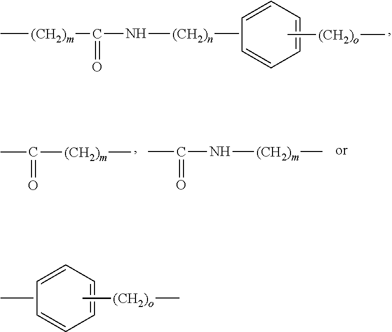
wherein m and o are integers from 0 to 6, and n is an integer from 1 to 6.

22. The compound of claim 21 wherein B is

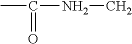

23. The compound of claim 22 were Y is lower alkylene.

24. The compound of claim 23 wherein X is 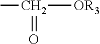
and R₃ is as above.

25. The compound of claim 22 where Y is 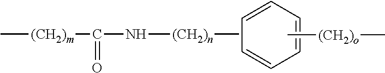
where m, n and o are as above.

26. A conjugate comprising a carrier linked to a ligand moiety of the formula. 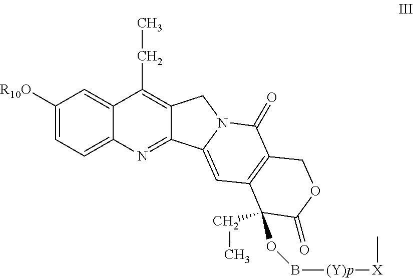
wherein X′ is —CH₂— or a linking group;
B is 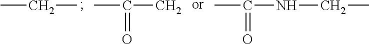
Y is an organic spacing group; R₁₀ is hydrogen or a hydrolyzable hydroxy protecting group and p is an integer from 0 to 1

27. The conjugate of claim 26, wherein the carrier contains a polyamine polymer with one of an amino group linked to said ligand.

28. The conjugate of claim 27, wherein p is 0.

29. The conjugate of claim 28, wherein X′ is 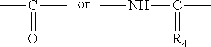
and R₄ is oxygen or sulfur.

30. The conjugate of claim 29, wherein p is 1.

31. The conjugate of claim 30, wherein Y is alkylene containing from 1 to 10 carbon atoms, 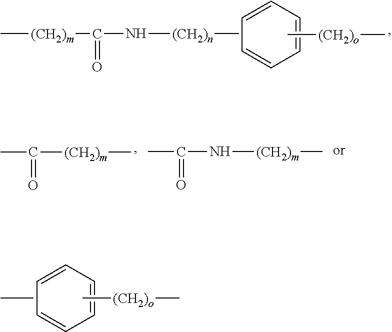
wherein m and o are integers from 0 to 6, and n is an integer from 1 to 6.

32. The conjugate of claim 31, wherein X′ is 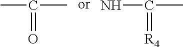
wherein R₄ is oxygen or sulfur.

33. The compound of claim 32 where X′ is 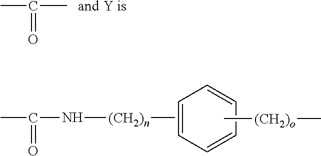
wherein n is as above and o is an integer of from 1 to 6.

34. The conjugate of claim 33, wherein said polyamine polymer is an immunogenic polymer.

35. The compound of claim 27 wherein Y is lower alkylene.

36. The compound of claim 35 wherein X′ is

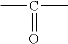

37. A kit for determining the presence of a pharmaceutically active metabolite of irinotecan and its pharmaceutically acceptable salts in a patient sample, said metabolite having the formula: 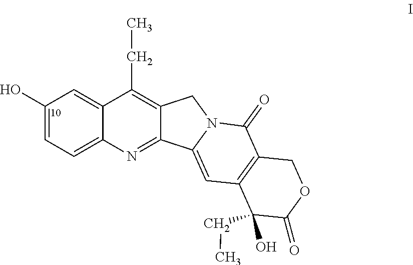
comprising reagents packaged in separate containers, one of the reagents being an antibody selectively reactive with said metabolite and its pharmaceutically acceptable salts and the other reagent being a conjugate of a carrier with a ligand of the formula: 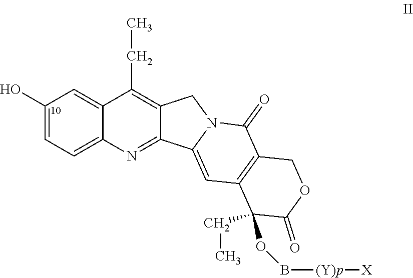
wherein B is 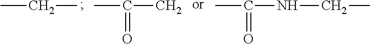
Y is an organic spacing group;
X is a terminal functional group capable of binding to said carrier, and
p is a an integer from 0 to 1.

38. The kit of claim 37 wherein said antibody is a monoclonal antibody.
